(19) United States
(12) Patent Application Publication
(10) Pub. No.: US 2026/0021278 A1
(43) Pub. Date: Jan. 22, 2026

(54) LOW-PROFILE STEERABLE CATHETER

(71) Applicant: Edwards Lifesciences Innovation (Israel) Ltd., Caesarea (IL)

(72) Inventor: Yaron Keidar, Kiryat Ono (IL)

(21) Appl. No.: 19/339,299

(22) Filed: Sep. 25, 2025

Related U.S. Application Data
(63) Continuation of application No. 17/680,975, filed on Feb. 25, 2022, now Pat. No. 12,440,648.
Application No. 17/680,975 is a continuation of application No. PCT/IL2020/050926, filed on Aug. 25, 2020.
(60) Provisional application No. 62/893,093, filed on Aug. 28, 2019.

(51) Int. Cl. A61M 25/01; A61M 29/00

(52) U.S. Cl. CPC A61M 25/0147; A61M 29/00; A61M 2025/015

(57) ABSTRACT

A steerable catheter comprises a flexible tube comprising a circumferential wall, along which a pull-wire extends, and that defines an elongate lumen from the tube's proximal portion to the tube's distal portion. A pull-ring, circumscribing the lumen at the distal portion, is coupled to the pull-wire and the wall such that pulling on the pull-wire bends the distal portion. The pull ring can be: (i) biased toward a first state in which the pull-ring is elliptical, and (ii) deformable to a second state in which the pull-ring has a lesser eccentricity. The tube can have a delivery state in which the pull-ring is in the first state and the distal portion's outer surface has a first surface eccentricity, and a working state in which the pull-ring is in the second state and the outer surface has a second, greater surface eccentricity. Other embodiments are also described.

### CROSS-REFERENCE TO RELATED APPLICATIONS

This application is a continuation of U.S. patent application Ser. No. 17/680,975, filed Feb. 25, 2022, which is a continuation of International Patent Application No. PCT/IL2020/050926, filed Aug. 25, 2020, which claims the benefit of U.S. Patent Application No. 62/893,093, filed Aug. 28, 2019, the entire disclosures all of which are incorporated by reference for all purposes.

### BACKGROUND

Steerable catheters can comprise a flexible tube, a pull-ring at a distal portion of the tube, and one or more pull-wires. The tube comprises a circumferential wall that defines an elongate lumen between the distal portion of the tube and a proximal portion of the tube. The pull-wires can extend, in association with the wall, from the proximal portion to the distal portion, where they can be coupled to a pull-ring.

Percutaneous catheters benefit from an outer diameter that is as small as possible for advancement of the catheter through the anatomy, and an inner diameter that is as large as possible for advancement of articles through the catheter. For some catheters, where the pull-wires are coupled to the pull-ring, the pull-wires may be arranged to overlap the pull-ring in order to maximize contact between these elements and increase the mechanical strength of the coupling. However, this may disadvantageously thicken the wall of the catheter, thereby increasing the outer diameter and/or reducing the inner diameter of the catheter.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Also, the features described can be combined in a variety of ways. Various features and steps as described elsewhere in this disclosure can be included in the examples summarized here.

A steerable catheter is provided, comprising a flexible tube with a pull-ring at a distal portion of the tube. At least one pull-wire extends, in association with a wall of the tube, between the distal portion and a proximal portion of the tube. The at least one pull-wire is coupled to the pull-ring, and is slidable with respect to the wall, such that proximal pulling (i.e., tensioning) of the at least one pull-wire bends the distal portion of the tube. The pull-ring is shaped and is elastically deformable such that, (i) during advancement of the catheter through the vasculature, an outer diameter of the tube is not adversely increased by the presence of the at least one pull-wire coupled to the pull-ring, and (ii) during advancement of an article through the lumen of the tube, an inner diameter of the tube is not adversely reduced by the need to accommodate the at least one pull-wire and the pull-ring within a small outer diameter of the tube.

The pull-ring may have (i) a first state in which the pull-ring is elliptical and has a first eccentricity, and (ii) a second state in which the pull-ring is elliptical and has a second eccentricity that is smaller than the first eccentricity. The pull-ring may be biased toward assuming the first state, and elastically deformable toward the second state.

There is therefore provided, in accordance with an application of the present invention, apparatus including a steerable catheter, the steerable catheter including a flexible tube, at least one pull-wire, and a pull-ring.

The flexible tube has a proximal portion (e.g., a proximal end) and a distal portion (e.g., a distal end), at least the distal portion being percutaneously advanceable into a subject. The flexible tube includes a circumferential wall that defines an elongate lumen between the proximal portion or proximal end and/or the distal portion or distal end.

The at least one pull-wire may extend, in association with the wall, from the proximal portion to the distal portion. The at least one pull-wire may be axially slidable with respect to the wall at least at the proximal portion. In some implementations, the at least one pull-wire extends within a secondary lumen extending within the wall of the tube.

At the distal portion of the tube, the pull-ring may be coupled to the wall such that the pull-ring circumscribes the lumen. The pull-ring is typically coupled to the at least one pull-wire, such that pulling on the at least one pull-wire bends the distal portion of the tube.

The pull-ring may have (i) a first state in which the pull-ring is elliptical and has a first eccentricity, and (ii) a second state in which the pull-ring is elliptical and has a second eccentricity that is smaller than the first eccentricity. The pull-ring may be biased toward assuming the first state and may be elastically deformable toward the second state. Herein, the term “eccentricity” means the numerical eccentricity as opposed to the linear eccentricity. The numerical eccentricity of a circle is zero. The numerical eccentricity of an ellipse which is not a circle is strictly greater than zero, but strictly less than 1. Therefore, when the pull-ring transitions from the first state to the second state, the pull-ring becomes less elliptical and more circular. More specifically, the pull-ring may have (i) a first state in which the pull-ring is noncircular elliptical, and has a minor diameter along a minor axis and a major diameter along a major axis, and (ii) a second state in which the pull-ring is circular and has a diameter that is equal to the major diameter of the pull-ring when the pull-ring is in the first state. Hence, the circumference of the pull-ring is smaller in the first state than in the second state. The first state of the expandable pull-ring is particularly advantageous during advancement of the catheter toward a target site, and the second state of the expandable pull-ring is particularly advantageous when the catheter has reached the target site.

In an application, the pull-ring, in at least the first state, defines a major axis and a minor axis, and the pull-ring is shaped to define a tension spring at each intersection of the major axis with the pull-ring.

In an application, the pull-ring is disposed at an axial site at the distal portion of the tube, the tube has an outer surface, and in the first state of the pull-wire, the outer surface of the tube at the axial site is circular in transverse cross-section. In some embodiments, an outer diameter of the tube at the axial site when the pull-ring is in the first state is equal to an outer diameter of the proximal portion of the tube.

In an application, the pull-ring is disposed at an axial site at the distal portion of the tube, the tube has an outer surface, and in the second state of the pull-wire, the outer surface of the tube at the axial site is non-circular elliptical in transverse cross-section. In some embodiments, a diameter of the noncircular elliptical pull-ring along the major axis when the pull-ring is in the first state is equal to a diameter of the circular pull-ring when the pull-ring is in the second state. Still preferably, an inner diameter of the tube at the axial site when the expandable pull-ring is in the second state is equal to an inner diameter of the proximal portion of the tube.

In an application, the tube is a first flexible tube, and defines, at the distal end, a distal opening out of the lumen, and the apparatus further includes a second flexible tube, advanceable distally through the lumen, past the pull-ring, and out of the distal opening, the second tube dimensioned such that advancement of the second tube through the lumen past the pull-ring elastically deforms the pull-ring toward the second state. The elastic deformation enlarges the circumference of the pull-ring as it transitions from the first state to the second state.

In an application, the at least one pull-wire is a first pull-wire, and the apparatus further includes a second pull-wire that extends, in association with the wall, from the proximal portion to the distal portion, and is axially slidable with respect to the wall at least at the proximal portion, and is coupled to the pull-ring opposite the first pull-wire. The first and second pull-wires can be configured to extend within the wall of the flexible tube.

In an application, the tube, at the proximal portion, is circular in transverse cross-section.

In an application, the pull-ring, in the second state, is circular. Hence, when the pull-ring is in the second state, the tube at the axial site is elliptical in transverse cross-section.

In an application, the pull-ring has (i) an inner surface that faces radially inward toward the lumen, and (ii) an outer surface that faces radially outward from the lumen, and the at least one pull-wire is attached to the outer surface of the pull-ring.

In an application:
the tube defines, at the distal portion and/or distal end, a distal opening out of the lumen,
the apparatus further includes a dilator, shaped to define:
a shaft, extending through the lumen;
a head; and
a neck, connecting the shaft to the head, and disposed within the pull-ring, the head extending distally away from the pull-ring and out of the distal opening, and
the dilator is dimensioned such that axial movement of the head through the pull-ring elastically deforms the pull-ring toward the second state. The dilator can be a transseptal dilator, but can also be used for other procedures.

In an application, the pull-ring is disposed at an axial site at the distal portion of the tube, and the neck is shaped to fit snugly within the lumen at the axial site. In particular, the shape of the neck can match the shape of the pull-ring in its first state.

In an application, the dilator is dimensioned such that the pull-ring resists axial movement of the neck away from the pull-ring.

In an application, the neck is noncircular in transverse cross-section.

In an application, the neck is noncircular elliptical in transverse cross-section. The transverse cross-section of the neck can correspond to the transverse cross-section of the pull-ring in its first state.

In an application, the head is conical.

In an application:
the head has a widest part, and tapers away from the widest part and from the neck, and
in at least one longitudinal cross-section, a narrowest portion of the neck is narrower than the widest part of the head.

In an application:
the pull-ring, in at least the first state, defines a major axis and a minor axis, and
the pull-wire is attached to the pull-ring at a circumferential position on the pull-ring that is within 10 degrees of the minor axis.

In an application, the circumferential position is on the minor axis, and the pull-wire is attached to the pull-ring at the circumferential position that is on the minor axis.

There is further provided, in accordance with an application of the present invention, a method, including:
transluminally advancing a distal portion of a flexible tube into a body chamber of a subject, and:
the tube includes a circumferential wall that defines an elongate lumen from a proximal portion of the tube to the distal portion,
the distal portion is shaped to define a distal opening out of the lumen, and
at the distal portion, an elliptical pull-ring is coupled to the wall such that the pull-ring circumscribes the lumen, the pull-ring being in a first state in which the pull-ring has a first eccentricity; and
while the distal portion is disposed in the body chamber, advancing an article through the lumen such that a distal end of the article passes the pull-ring and elastically deforms the pull-ring into a second state in which the pull-ring has a second eccentricity that is smaller than the first eccentricity.

In an application, advancing the distal portion of the flexible tube includes advancing the distal portion of the flexible tube while the distal portion is circular in transverse cross-section.

In an application, advancing the article through the lumen includes advancing the article through the lumen such that the distal end of the article passes the pull-ring and elastically deforms the distal portion of the flexible tube to become noncircular elliptical in transverse cross-section.

In an application, advancing the article through the lumen includes advancing the article through the lumen such that the distal end of the article passes the pull-ring and elastically deforms the pull-ring into a second state in which the pull-ring is circular in transverse cross-section.

In an application, the flexible tube is a first flexible tube, the article includes a second flexible tube, and advancing the article through the lumen includes advancing the second flexible tube through the lumen.

In an application, advancing the distal portion includes advancing the distal portion while a transseptal dilator is arranged with respect to the tube such that a head of the dilator extends out of the distal opening, a neck of the dilator is disposed within the pull-ring, and a shaft of the dilator extends from the neck proximally through the lumen.

In an application, the method further includes, while the distal portion is disposed in the body chamber, and prior to advancing the article through the lumen, proximally withdrawing the dilator such that movement of the head through the pull-ring elastically deforms the pull-ring toward the second state.

This method can be performed on a living animal or on a simulation, such as on a cadaver, cadaver heart, simulator (e.g. with the body parts, tissue, etc. being simulated), etc.

There is further provided, in accordance with an application of the present invention, a system and/or apparatus including a steerable catheter, the steerable catheter including:
a flexible tube:
having a proximal portion and a distal portion, at least the distal portion being percutaneously advanceable into a subject, and
including a circumferential wall that defines an elongate lumen between the proximal portion and the distal portion;
a pull-ring at the distal portion of the tube, coupled to the wall such that the pull-ring circumscribes the lumen;
a first bundle of pull-wires, extending, from the proximal portion to the distal portion, in association with the wall on a first lateral side of the lumen, and coupled to the pull-ring such that pulling on the first bundle bends the distal portion of the tube in a first direction; and
a second bundle of pull-wires, extending, from the proximal portion to the distal portion, in association with the wall on a second lateral side of the lumen, and coupled to the pull-ring opposite the first bundle such that pulling on the second bundle bends the distal portion of the tube in a second direction that is opposite to the first direction.

In an application, each of the pull-wires of the first bundle and of the second bundle has insufficient tensile strength, on its own, to bend the distal portion of the tube when pulled.

In an application, for each of the bundles, the pull-wires of the bundle extend, from the proximal portion to the distal portion, parallel with each other.

In an application, for each of the bundles, the pull-wires of the bundle extend, from the proximal portion to the distal portion, twisted around each other.

In an application, for each of the first bundle and the second bundle, at a distal end of the bundle, the pull-wires of the bundle are untwisted and the bundle is flattened such that each of the pull-wires of the bundle contacts the pull-ring.

The present invention will be more fully understood from the following detailed description of applications thereof, taken together with the drawings, in which:

### BRIEF DESCRIPTION OF THE DRAWINGS

FIG. 1 is a schematic illustration of a prior art steerable catheter comprising a flexible tube, one or more pull-wires, and a pull-ring;

FIGS. 2A-C are schematic illustrations of a steerable catheter comprising a flexible tube, one or more bundles of pull-wires, and a pull-ring, in accordance with some applications of the invention; and

FIGS. 3A-B, 4, and 5A-D are schematic illustrations of a steerable catheter, and a system comprising the catheter, in accordance with some applications of the invention.

### DETAILED DESCRIPTION OF EMBODIMENTS

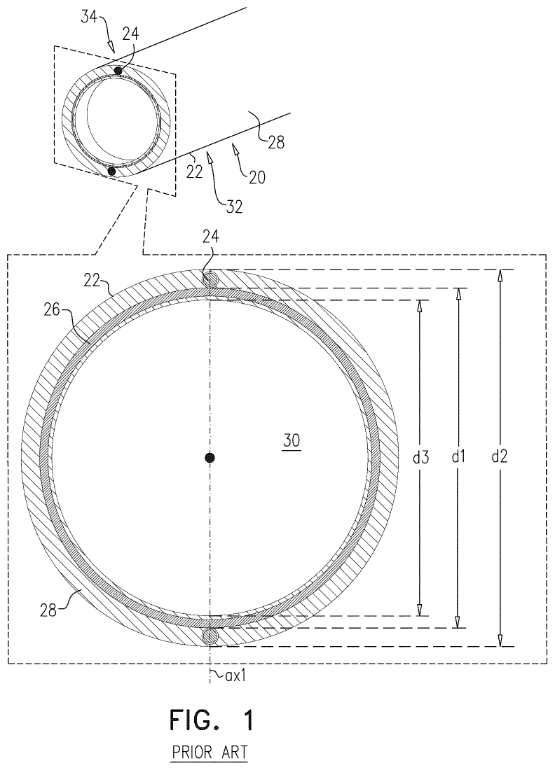

Each of the catheters described herein, including the prior art catheter of FIG. 1, comprises a flexible tube, one or more pull-wires, and a pull-ring. Each catheter has a proximal portion and a distal portion, at least the distal portion being percutaneously (e.g., transluminally) advanceable into a subject. The tube comprises a circumferential wall that defines an elongate lumen between the proximal portion of the catheter and the distal portion of the catheter (e.g., between a proximal end and a distal end of the catheter). The pull-ring is attached to the tube at an axial site of the tube, within the distal portion of the tube (e.g., at or close to a distal end of the tube), where it is coupled to the wall such that the pull-ring circumscribes the lumen. For example, the pull-ring may be embedded in the wall. Each pull-wire extends, in association with the wall, from the proximal portion of the catheter to the distal portion of the catheter (e.g., to the axial site), where it is coupled to the pull-ring. Tensioning a given pull-wire (e.g., by pulling proximally from a proximal end) bends the distal portion of the tube, e.g., toward the side on which the given pull-wire is disposed. To facilitate this, each pull-wire may be axially slidable with respect to the wall (at least at the proximal portion).

Typically, the pull-wires are embedded within the wall of the catheter (e.g., slidably disposed within a secondary lumen within the wall). The pull-ring can also be embedded within the wall, such that the material of the wall covers both an inner surface and an outer surface of the pull-ring. The wall can comprise multiple layers (e.g., of one or more polymers) that are fused together. At the axial site at which the pull-ring is disposed, the wall is typically sufficiently thick to cover the pull-ring and the pull-wire(s).

Reference is made to FIG. 1, which is a schematic illustration of a prior art steerable catheter 20 comprising a flexible tube 22, one or more (e.g., two) pull-wires 24, and a pull-ring 26. Catheter 20 (e.g., tube 22 thereof) has a proximal portion (not shown) and a distal portion 32, at least the distal portion being percutaneously (e.g., transluminally) advanceable into a subject. Tube 22 comprises a circumferential wall 28 that defines an elongate lumen 30 between the proximal portion of catheter 20 and distal portion 32.

Each pull-wire 24 extends, in association with wall 28, from the proximal portion of catheter 20 to distal portion 32, and is axially slidable with respect to the wall at least at the proximal portion.

Pull-ring 26 is disposed at an axial site 34 of tube 22, within distal portion 32, where it is coupled to wall 28 such that the pull-ring circumscribes lumen 30. For example, pull-ring 26 may be embedded in wall 28. Pull-ring 26 is coupled to each pull-wire 24 at axial site 34 such that pulling on a given pull-wire bends the distal portion of the tube, e.g., toward the side on which the given pull-wire is disposed.

Each pull-wire 24 is disposed on a transverse axis ax1 of catheter 20. Axis ax1 typically passes through the pull-wire and a central longitudinal axis of the catheter. For applications in which catheter 20 comprises two pull-wires 24, both of the pull-wires are disposed on axis ax1, diametrically opposite each other. That is, axis ax1 is defined, on a transverse cross-section of catheter 20, between the pull-wires, and the central longitudinal axis of the catheter typically also lies on axis ax1. Because axis ax1 is defined between the pull-wires, axis ax1 also can define the steering plane of catheter 20.

It is often advantageous for a percutaneous catheter to have a small outer diameter, and advantageous to minimize the difference between the inner diameter of the catheter and the outer diameter of the catheter, in order to (i) enable advancement of the catheter through small apertures while (ii) maximizing the space available within the catheter for articles (e.g., implants and/or tools) to pass through. Pull-ring 26 is circular (when viewed in a transverse cross-section of catheter 20) and has an outer surface that defines an outer diameter d1 of the pull-ring. The difference between the outer diameter of catheter 20 (e.g., tube 22) and the inner diameter of the catheter (i.e., a width of lumen 30) is at least partly dependent on a wall thickness of the pull-ring. However, this difference is also partly dependent on the thickness (e.g., diameter) of pull-wires 24, which are often attached to pull-ring 26 at the outer surface of the pull-ring. Pull-wires 24 must withstand the forces (e.g., tension) that will be applied in order to bend tube 22, and this strength is typically achieved, inter alia, by the pull-wires being sufficiently thick. Therefore, at least on transverse axis ax1, catheter 20 (e.g., tube 22) has an outer diameter d2 that is greater than its inner diameter, typically by at least twice the wall thickness of pull-ring 26 plus the thickness of each pull-wire 24 disposed on axis ax1. The thickness of the material of wall 28 also generally contributes to the difference between the outer and inner diameters.

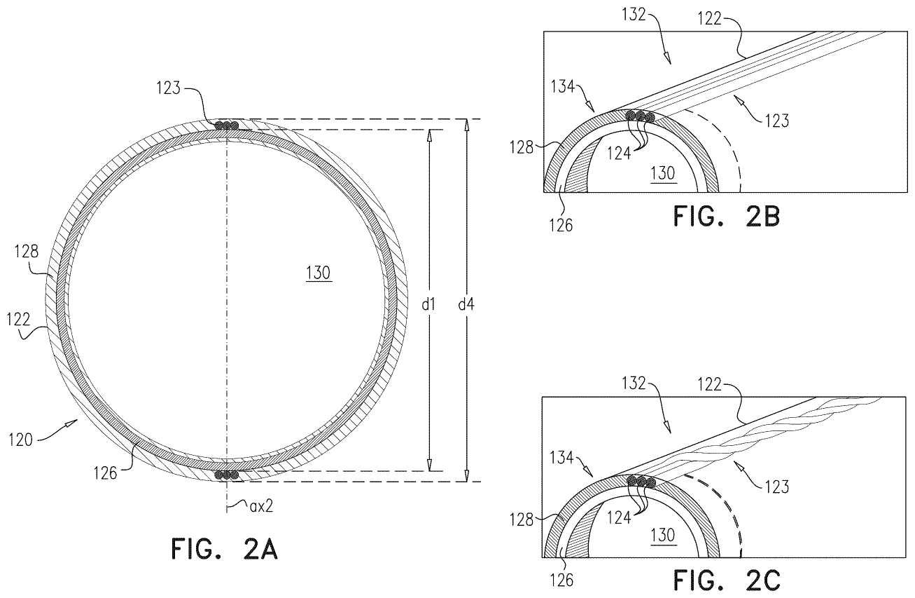

Reference is made to FIGS. 2A-C, which are schematic illustrations of a steerable catheter 120 comprising a flexible tube 122, one or more (e.g., two, three, four, etc.) bundles 123 of pull-wires 124, and a pull-ring 126, in accordance with some applications of the invention. For some applications, pull-ring 126 is identical to pull-ring 26.

Catheter 120 (e.g., tube 122 thereof) has a proximal portion (not shown) and a distal portion 132, at least the distal portion being percutaneously (e.g., transluminally) advanceable into a subject. Tube 122 comprises a circumferential wall 128 that defines an elongate lumen 130 between the proximal portion of catheter 120 and distal portion 132 (e.g., between a proximal end and a distal end of the catheter).

Each bundle 123 extends, in association with wall 128, from the proximal portion of catheter 120 to distal portion 132, and is axially slidable with respect to the wall at least at the proximal portion.

Pull-ring 126 is disposed at an axial site 134 of tube 122, within distal portion 132, where it is coupled to wall 128 such that the pull-ring circumscribes lumen 130. For example, pull-ring 126 may be embedded in wall 128. Pull-ring 126 is coupled to each bundle 123 at axial site 134 such that pulling on a given bundle bends the distal portion of the tube, e.g., toward the side on which the given bundle is disposed.

Each bundle 123 is disposed on a transverse axis ax2 of catheter 120. Axis ax2 typically that passes through the bundle and a central longitudinal axis of the catheter. For applications in which catheter 120 comprises two bundles 123, both of the bundles are typically disposed on axis ax2, diametrically opposite each other. That is, axis ax2 is typically defined, on a transverse cross-section of catheter 120, between the bundles, and typically the central longitudinal axis of the catheter intersects axis ax2.

Each pull-wire 124 is thinner, and typically weaker, than pull-wire 24. For some applications, each pull-wire 124 has insufficient tensile strength, on its own, to bend the distal portion of the tube 122 when pulled. However, collectively, the plurality of pull-wires 124 in each bundle 123 provide the bundle with sufficient strength to bend the tube. At least at axial site 134 the pull-wires 124 of each bundle 123 are disposed adjacent to each other, circumferentially arranged around a small portion of pull ring 126, such that each of the pull-wires is flat against the pull-ring.

Because each pull-wire 124 is thinner than pull-wire 24, at least at axial site 134 bundles 123 have a flattened profile compared to pull-wire 24, the bundles being thinner on the transverse axis ax2 on which they are disposed. Therefore, at least at the axial site 134 at which the pull-ring is disposed, the difference between an outer diameter d4 of catheter 120 and an inner diameter of the catheter (at site 134) is advantageously smaller than the difference between the outer and inner diameters of catheter 20. In the example shown, the outer diameter of pull-ring 126 is d1 (i.e., the same as that of pull-ring 26), and therefore it is possible for outer diameter d4 of catheter 120 to be smaller than outer diameter d2 of catheter 20.

For some applications, and as shown in FIG. 2B, the pull-wires 124 of each bundle extend, in parallel with each other, between the proximal portion of the tube and distal portion 132. For some applications, and as shown in FIG. 2C, the pull-wires 124 of each bundle are twisted (e.g., braided) around each other between the proximal portion and distal portion 132—but nonetheless, at axial site 134, the bundle is flattened such that the pull-wires 124 of each bundle 123 are disposed adjacent to each other, circumferentially arranged around a small portion of pull ring 126, such that each of the pull-wires is flat against the pull-ring.

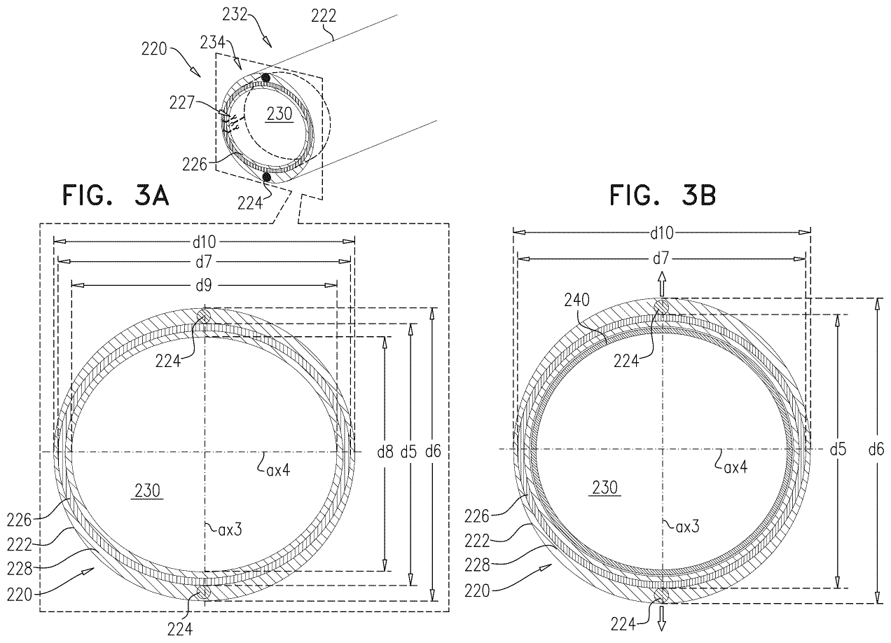

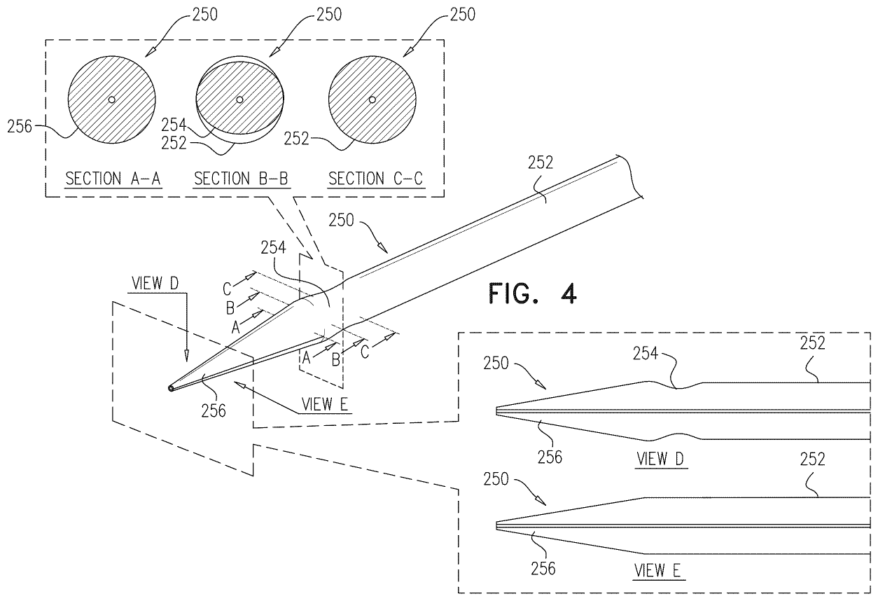

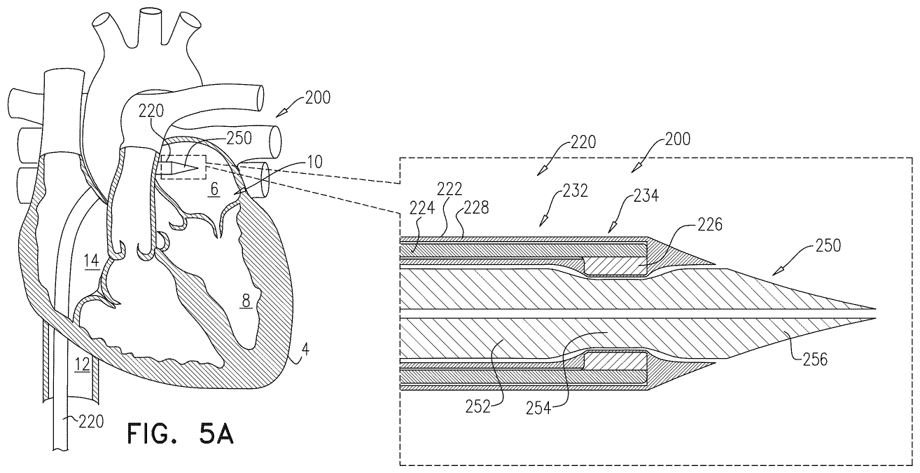

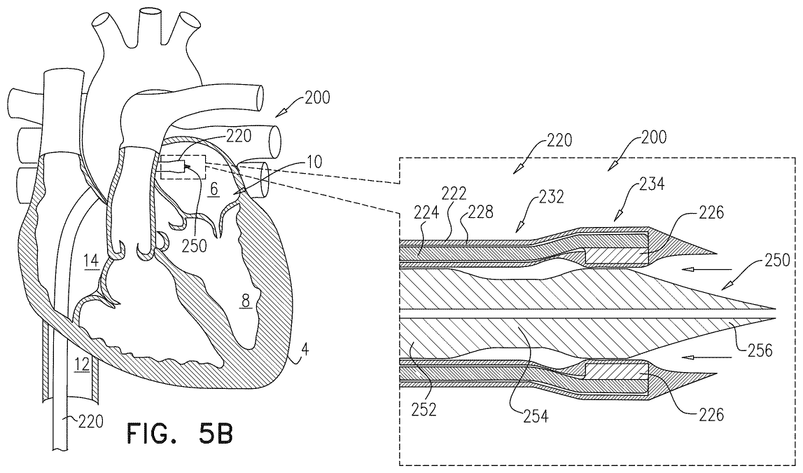

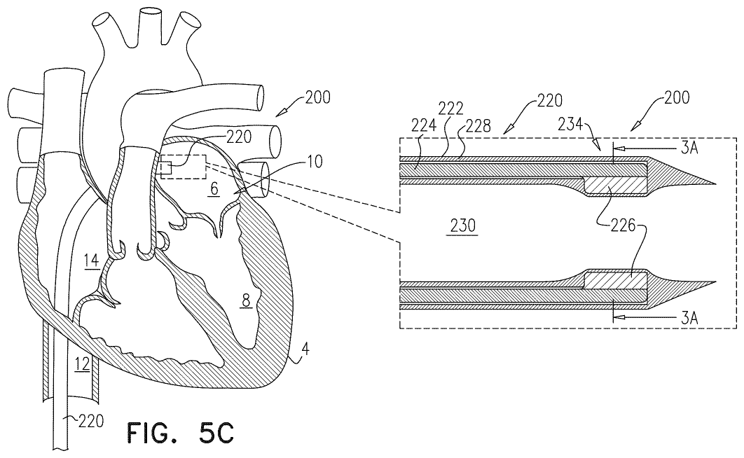

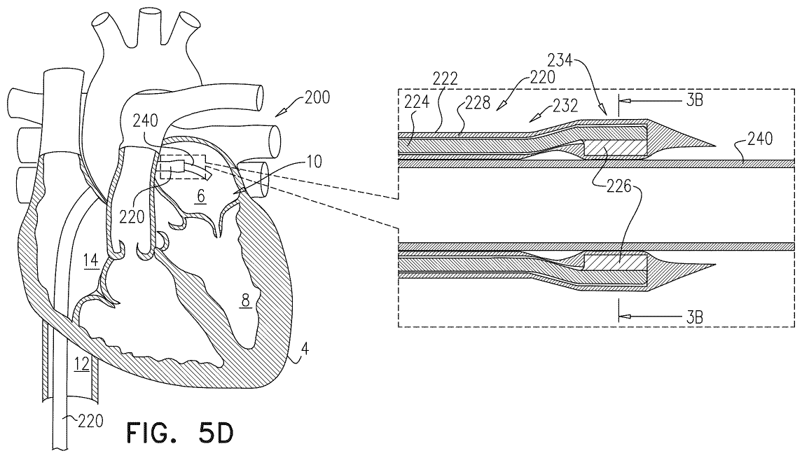

Reference is made to FIGS. 3A-B, 4, and 5A-D, which are schematic illustrations of a steerable catheter 220, and a system 200 comprising catheter 220, in accordance with some applications of the invention. Like catheter 120, catheter 220 is configured to provide, at its distal portion (e.g., at its pull-ring) an outer diameter that is sufficiently small for percutaneous (e.g., transluminal) advancement, and an inner diameter that is sufficiently large for advancement of articles (e.g., implants and/or tools) therethrough. In catheter 220, this is achieved by providing the small outer diameter specifically during percutaneous advancement and providing the large inner diameter specifically during advancement of implants and/or tools therethrough. During percutaneous advancement of the catheter, the large inner diameter is sacrificed in order to provide the small outer diameter. Once the distal portion of the catheter has reached the target site, the small outer diameter is sacrificed in order to provide the large inner diameter for advancement of articles through the catheter.

Catheter 220 comprises a flexible tube 222, one or more (e.g., two, three, four, etc.) pull-wires 224, and a pull-ring 226. Catheter 220 (e.g., tube 222 thereof) has a proximal portion (not shown) and a distal portion 232, at least the distal portion being percutaneously (e.g., transluminally) advanceable into a subject. Tube 222 comprises a circumferential wall 228 that defines an elongate lumen 230 between the proximal portion of catheter 220 and distal portion 232 (e.g., between a proximal end and a distal end of the catheter).

Each pull-wire 224 extends, in association with wall 228, from the proximal portion of catheter 220 to distal portion 232 and is axially slidable with respect to the wall at least at the proximal portion.

Pull-ring 226 is disposed at an axial site 234 of tube 222, within distal portion 232, where it is coupled to wall 228 such that the pull-ring circumscribes lumen 230. For example, pull-ring 226 may be embedded in wall 228. Pull-ring 226 is coupled to each pull-wire 224 at axial site 234 such that pulling on a given pull-wire bends the distal portion of the tube, e.g., toward the side on which the given pull-wire is disposed. Pull-ring 226 has an inner surface that faces radially inward toward the lumen, and an outer surface that faces radially outward from the lumen, and the one or more pull-wires are often attached to the outer surface of the pull-ring. The one or more pull-wires can generally also be attached to the inner surface of the pull-ring, or at least one of the pull-wires can be attached to the outer surface and the other of the pull-wires can be attached to the inner surface.

Each pull-wire 224 can be disposed on a transverse axis ax3 of catheter 220. Axis ax3 typically passes through the pull-wire and a central longitudinal axis of the catheter (e.g., axis ax3 is perpendicular to central longitudinal axis of the catheter). For applications in which catheter 220 comprises two pull-wires 224, both of the pull-wires are often disposed on axis ax3, diametrically opposite each other. That is, axis ax3 is often defined, on a transverse cross-section of catheter 220, between the pull-wires, and the central longitudinal axis of the catheter intersects axis ax3.

In a first state of pull-ring 226 (FIG. 3A), the pull-ring is elliptical and has a first eccentricity, and in a second state of the pull-ring, the pull-ring is elliptical and has a second eccentricity that is smaller than the first eccentricity (FIG. 3B). For example, and as shown, in transverse cross-section, pull-ring 226 may be noncircular elliptical in the first state, and circular in the second state.

Pull-ring 226 is biased toward assuming the first state and is elastically deformable toward the second state. Therefore, the first state may be considered to be a resting or default state.

In the first state of pull-ring 226, the noncircular elliptical shape of the pull-ring defines a major axis ax4 and defines axis ax3 as a minor axis. That is, pull-wires 224 can be disposed on minor axis ax3 of the noncircular elliptically-shaped pull-ring, at respective circumferential positions opposite each other on the pull-ring. However, for some applications pull-wires 224 are disposed partway around the pull-ring from axis ax3, such as within 10 degrees of the minor axis.

In its first state, a minor outer diameter d5 of pull-ring 226 along minor axis ax3 is smaller than a major outer diameter d7 of the pull-ring along major axis ax4. When pull-ring 226 is in its first state, an outer diameter d6 of catheter 220 along minor axis ax3 can be equal to an outer diameter d10 of catheter 220 along major axis ax4, so that catheter 220 has a circular cross-section at axial site 234. In this way, catheter 220 may have a smooth outer surface along its entire length.

It is to be noted that minor axis ax3 lies on (or defines) the steering plane of catheter 220, and major axis ax4 is orthogonal to the steering plane of the catheter.

For some applications, pull-ring 226 comprises or is shaped to define a tension spring 227 at each intersection of major axis ax4 with the pull-ring, in order to bias the pull-ring toward assuming the first state. Tension spring 227 allows elastic expansion of pull-ring 226. For example, tension spring 227 allows pull-ring 226 to transition from its first state to its second state. More specifically, tension spring 227 allows noncircular pull-ring 226 to assume a circular shape, e.g., by allowing outer diameter d5 of pull-ring 226 along minor axis ax3 to expand to outer diameter d7 of pull-ring 226 along major axis ax4. When pull-ring 226 is in its second state, catheter 220 may have the same inner diameter, e.g., inner diameter d9 along major axis ax4 in the first state of pull-ring 226, along its entire length, which facilitates insertion of catheters or articles through lumen 230. In such applications, when pull-ring 226 transitions from its first state to its second state, the transverse cross-section of catheter 220 at axial site 234 transitions from a circular to a noncircular shape. In other words, tension spring 227 is configured to allow a first half of pull-ring 226 to move radially away from a second half of pull-ring 226. Tension spring 227 may be realized by one or more (e.g., three) slits in pull-ring 226 at each intersection of major axis ax4 with pull-ring 226 (FIG. 3A). However, other configurations of tension springs 227 are also possible. For example, a tension spring 227 could be formed by a corrugated portion of pull-ring 226. It will be understood that tension springs 227 can be located at other positions along the circumference of the pull-ring 226, either in lieu of or in addition to tension springs 227 located at the two intersections of major axis ax4 with pull-ring 226. Circumferential wall 228 can be made of a material, typically a polymer, allowing elastic expansion of tube 222 at least at axial site 234. In applications where circumferential wall 228 comprises a braided layer, e.g. a steel braid, axial site 234 may be left unbraided to allow the expansion of tube 222. Optionally, the material of the braid at least at the axial site 234 can be an elastomer, such as Nylon. For some applications, pull-ring 226 does not comprise a discrete spring, but is generally shape-set to be biased toward assuming the first state. In other applications, pull-ring 226 is made of an elastically extensible material. Generally, the circumference of the pull-ring is smaller in the first state than in the second state.

For a catheter that comprises two pull-wires opposite each other, at the axial site of the pull-ring, and on the minor (or transverse) axis ax3 at which the pull-wires are coupled to the pull-ring of the catheter (i.e., on the steering plane of the catheter), the outer diameter of the catheter is at least the sum of (i) the outer diameter of the pull-ring, and (ii) the thickness of each pull-wire. This outer diameter would be greater than the outer diameter required on major axis ax4 (which is orthogonal to the steering plane) since none of the pull-wires lie on major axis ax4. That is, although on major axis ax4 it may be possible for the catheter to have an outer diameter that is insignificantly larger than the outer diameter of the pull-ring, on minor axis ax3 that lies on the steering plane the outer diameter of the catheter must be greater by at least the thickness of both pull-wires. Therefore, if the anatomy through which such a catheter must be advanced requires the outer diameter of the catheter to be no greater than a given outer diameter, the inner diameter of the catheter must disadvantageously be smaller than the given outer diameter by at least the thickness of the pull-wires and the thickness of the pull-ring.

For catheter 220, the noncircular elliptical shape of pull-ring 226 in the first state facilitates a reduced outer diameter d6 of the catheter on minor axis ax3 (i.e., on the steering plane), compared to a similar catheter with a circular pull-ring (FIG. 3A) having outer diameter d7. In this way, outer diameter d6 on minor axis ax3 may be equal to an outer diameter d10 of the catheter on major axis ax4. For some applications, in the first state, although pull-ring 226 is noncircular elliptical in transverse cross-section, the outer surface of tube 222 at axial site 234 is circular in transverse cross-section. Although, in the first state, at axial site 234 an inner diameter d8 on minor axis ax3 is smaller than an inner diameter d9 on major axis ax4, this is not important during advancement of the catheter through the anatomy because lumen 230 is not being used. FIG. 5A shows catheter 220 having been advanced transluminally, via inferior vena cava 12, through right atrium 14 of heart 4, and transseptally into left atrium 6, while in the first state of the catheter. (FIG. 5A also shows a dilator 250, discussed in more detail hereinbelow. FIG. 5C shows the same state of catheter 220, in the absence of the dilator.)

Once distal portion 232 of catheter 220 has reached the target site, one or more articles, such as an inner catheter 240 (typically comprising a second flexible tube), can be advanced through lumen 230, past pull-ring 226. If the article being advanced has an outer diameter that is larger than inner diameter d8 on minor axis ax3, it pushes radially outward on the inner surface of the catheter as it passes the pull-ring, enlarging inner diameter d8, with pull-ring 226 responsively elastically deforming toward its second state (FIG. 3B). Lumen 230 can thereby accommodate an article that has an outer diameter that is greater than inner diameter d8 on minor axis ax3 was in the first state. FIG. 5D shows catheter 220, in its second state, with inner catheter 240 having been advanced through lumen 230, and extending out of the distal end of catheter 220.

There is therefore provided, in accordance with some applications of the invention, a method comprising:
transluminally advancing a distal portion of a flexible tube into a body chamber (such as a heart chamber, e.g., left atrium 6), while an elliptical pull-ring, at the distal portion, is in a first state in which the pull-ring has a first eccentricity; and
while the distal portion is disposed in the body chamber, advancing an article through the flexible tube past the pull-ring such that the article elastically deforms the pull-ring into a second state in which the pull-ring has a second eccentricity that is smaller than the first eccentricity.

Often, in the second state, outer diameter d6 on minor axis ax3 is greater than outer diameter d10 on major axis ax4. For example, in the second state, although pull-ring 226 may be circular in transverse cross-section, the outer surface of tube 222 at axial site 234 may be noncircular elliptical in transverse cross-section. Although, in the second state, outer diameter d6 on minor axis ax3 is greater than outer diameter d10 on major axis ax4, this is not important during advancement of catheter 240 through lumen 230 because distal portion 232 of catheter 220 is already disposed at the target site (e.g., within left atrium 6).

Despite the configuration of distal portion 232 (e.g., axial site 234) of catheter 220 described hereinabove, the proximal portion of catheter 220 may be circular in transverse cross-section.

FIG. 4 shows a transseptal dilator 250, which is another component of system 200, in accordance with some applications of the invention. Dilator 250 comprises (or is shaped to define) a shaft 252, a neck 254, and a head 256 connecting the shaft to the head. Head 256 is typically conical. Often, and as shown, in at least one longitudinal cross-section (e.g., view D), neck 254 is narrower than at least a widest portion of head 256. For some such applications, and as shown in that longitudinal cross-section, neck 254 is also narrower than shaft 252. For some applications, and as shown, neck 254 is noncircular in transverse cross-section. For example, neck 254 may be noncircular elliptical in transverse cross-section. Neck 254 may have the shape of a cylinder that has been squeezed from two opposite sides.

Head 256 can be configured to taper away (i.e., distally away) from its widest part and from neck 254.

As shown in FIG. 5A, during transluminal advancement of system 200, (i) neck 254 is disposed at axial site 234, and is therefore disposed within pull-ring 226, and (ii) head 256 extends distally away from the pull-ring and out of the distal opening of catheter 220 (e.g., of tube 222 thereof). In this arrangement, pull-ring 226 is in its first state. Neck 254 can be configured to fit snugly within lumen 230 at axial site 234, e.g., by having a shape that mates with the inner surface of catheter 220, the shape of the inner surface at least partly resulting from the shape of pull-ring 226.

Dilator 250 can be dimensioned such that pull-ring 226 resists axial movement of neck 254 away from the pull-ring, e.g., resisting proximal withdrawal of dilator 250 through lumen 230.

As also shown in FIG. 5A, dilator 250 is used to facilitate transseptal advancement of catheter 220 through the interatrial septum (i.e., from right atrium 14 into left atrium 6). This is performed while pull-ring 226 is in its first state. Besides transseptal advancement, other delivery approaches are also possible.

Once distal portion 232 of catheter 220 is within left atrium 6, dilator 250 is withdrawn (FIG. 5B). The resulting axial movement of head 256 through pull-ring 226 elastically deforms the pull-ring toward its second state. Once head 256 has passed pull-ring 226, the pull-ring typically returns automatically toward its first state (FIG. 5C).

Subsequently, a second catheter 240 (and/or another article, such as a tool or an implant) is advanced through lumen 230 of catheter 220 (FIG. 5D). As described hereinabove, pull-ring 226 facilitates this by responsively elastically deforming toward its second state.

Distal portion 232 of catheter 220 may be deflected (i.e., bent) using pull-wires 224, before and/or after catheter 240 has been advanced through lumen 230, in order to guide the procedure.

At the end of the procedure, catheter 240 is withdrawn, allowing pull-ring 226 to return to its first state, and therefore allowing catheter 220 to be transluminally withdrawn while the pull-ring is in its first state.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, techniques, methods, operations, steps, etc. described or suggested herein can be performed on a living animal or on a non-living simulation, such as on a cadaver, cadaver heart, simulator (e.g. with the body parts, tissue, etc. being simulated), etc.

We claim:

1. An apparatus comprising a steerable catheter, the steerable catheter comprising:
a flexible tube:
having a proximal portion and a distal portion, at least the distal portion being percutaneously advanceable into a subject, and
comprising a circumferential wall that defines an elongate lumen between the proximal portion and the distal portion;
a pull-wire, extending, in association with the wall, from the proximal portion to the distal portion; and
a pull-ring:
at the distal portion of the tube, coupled to the wall such that the pull-ring circumscribes the lumen,
coupled to the pull-wire, such that pulling on the pull-wire bends the distal portion of the tube,
having (i) a first state in which the pull-ring is elliptical and has a first pull-ring eccentricity, and (ii) a second state in which the pull-ring is elliptical and has a second pull-ring eccentricity that is smaller than the first pull-ring eccentricity,
biased toward assuming the first state, and
elastically deformable toward the second state,
wherein the distal portion of the flexible tube has an outer surface, and has:
a delivery state in which the pull-ring is in the first state and the outer surface has a first surface eccentricity, and
a working state in which the pull-ring is in the second state and the outer surface has a second surface eccentricity that is greater than the first surface eccentricity.

2. The apparatus according to claim 1, wherein:
the distal portion of the tube defines a first outer diameter along a minor axis of the pull-ring;
the distal portion of the tube further defines a second outer diameter along a major axis of the pull-ring; and
the tube is configured such that:
while the pull-ring is in the first state, the first outer diameter is equal to the second outer diameter, and
while the pull-ring is in the second state, the first outer diameter is greater than the second outer diameter.

3. The apparatus according to claim 1, wherein:
the pull-ring, in at least the first state, defines a major axis and a minor axis, and
the pull-ring is shaped to define a tension spring at each intersection of the major axis with the pull-ring.

4. The apparatus according to claim 1, wherein:
the pull-ring is disposed at an axial site at the distal portion of the tube,
the tube has an outer surface, and
in the first state, the outer surface of the tube at the axial site is circular in transverse cross-section.

5. The apparatus according to claim 1, wherein:
the pull-ring is disposed at an axial site at the distal portion of the tube,
the tube has an outer surface, and
in the second state, the outer surface of the tube at the axial site is non-circular elliptical in transverse cross-section.

6. The apparatus according to claim 1, wherein:
the pull-wire is a first pull-wire, and
the apparatus further comprises a second pull-wire:
extending, in association with the wall, from the proximal portion to the distal portion,
axially slidable with respect to the wall at least at the proximal portion, and
coupled to the pull-ring opposite the first pull-wire.

7. The apparatus according to claim 1, wherein the tube, at the proximal portion, is circular in transverse cross-section.

8. The apparatus according to claim 1, wherein the pull-ring, in the second state, is circular.

9. The apparatus according to claim 1, wherein:
the pull-ring has (i) an inner surface that faces radially inward toward the lumen, and (ii) an outer surface that faces radially outward from the lumen, and
the pull-wire is attached to the outer surface of the pull-ring.

10. The apparatus according to claim 1, further comprising an article:
having an outer diameter that is greater than an inner diameter of the distal portion of the tube while the pull-ring is in the first state, and
configured to, while the distal portion of the tube is disposed in a body chamber, deform the pull-ring toward the second state by the article being slid axially through the lumen and the pull-ring, thereby transitioning the distal portion of the tube from the delivery state to the working state.

11. The apparatus according to claim 10, wherein:
the tube is a first flexible tube, and defines, at the distal portion, a distal opening out of the lumen, and
the article comprises a second flexible tube, advanceable distally through the lumen, past the pull-ring, and out of the distal opening, the second flexible tube dimensioned such that advancement of the second flexible tube through the lumen past the pull-ring elastically deforms the pull-ring toward the second state.

12. The apparatus according to claim 1, wherein:
the tube defines, at the distal portion, a distal opening out of the lumen,
the apparatus further comprises a transseptal dilator, shaped to define:
a shaft, extending through the lumen;
a head; and
a neck, connecting the shaft to the head, and disposed within the pull-ring, the head extending distally away from the pull-ring and out of the distal opening, and
the dilator is dimensioned such that axial movement of the head through the pull-ring elastically deforms the pull-ring toward the second state.

13. The apparatus according to claim 12, wherein the pull-ring is disposed at an axial site at the distal portion of the tube, and the neck fits snugly within the lumen at the axial site.

14. The apparatus according to claim 12, wherein the dilator is dimensioned such that the pull-ring resists axial movement of the neck away from the pull-ring.

15. The apparatus according to claim 12, wherein the neck is noncircular in transverse cross-section.

16. The apparatus according to claim 15, wherein the neck is elliptical in transverse cross-section.

17. The apparatus according to claim 12, wherein the head is conical.

18. The apparatus according to claim 17, wherein:
the head has a widest part, and tapers away from the widest part and from the neck, and
in at least one longitudinal cross-section, a narrowest portion of the neck is narrower than the widest part of the head.

19. The apparatus according to claim 1, wherein:
the pull-ring, in at least the first state, defines a major axis and a minor axis, and
the pull-wire is attached to the pull-ring at a circumferential position on the pull-ring that is within 10 degrees of the minor axis.

20. The apparatus according to claim 19, wherein the circumferential position is on the minor axis, and the pull-wire is attached to the pull-ring at the circumferential position that is on the minor axis.
